(19) United States
(12) Patent Application Publication
(10) Pub. No.: US 2009/0143728 A1
(43) Pub. Date: Jun. 4, 2009

(54) BALLOON CATHETER WITH SAFETY FEATURE

(75) Inventor: Allen J. Tower, North Lawrence, NY (US)

Correspondence Address: Hiscock & Barclay, LLP, One Park Place, 300 South State Street, Syracuse, NY, 13202-2078, US

(73) Assignee: Numed, Inc., Nicholville, NY (US)

(21) Appl. No.: 11/947,870

(22) Filed: Nov. 30, 2007

(51) Int. Cl. A61M 25/10

(52) U.S. Cl. 604/101.02

(57) ABSTRACT

A balloon catheter having a safety feature for protecting a patient during a medical procedure in the event the inflated balloon ruptures during the procedure. The catheter contains an outer balloon attached to the distal end of an insertion tube and a second inner balloon totally enclosed within the outer balloon. A vacuum is drawn on the outer balloon to evacuate the interior of the balloon and the inner balloon is then inflated to a desired operating pressure. In the event the inner balloon ruptures while inflated, the inflation fluid is fully contained within the outer balloon as well as any inner balloon fragments that are released due to the rupture.

### FIELD OF THE INVENTION

This invention relates to a balloon catheter generally and, in particular, to a safety feature that is associated with a balloon catheter for preventing harm to a patient in the event the balloon ruptures during a percutaneous medical procedure.

### BACKGROUND OF THE INVENTION

Balloon catheters have been known and used in the art for a number of years. Advances in the art, have considerably reduced the risk of the balloon becoming dislodged from the catheter during a medical procedure. Another risk associated with balloon catheters is the danger of the balloon rupturing inside a patient while inflated. Although the risk of rupture is extremely small, it can be dangerous if the rupture occurs within a vital organ, such as the heart or the like. Pressures of between 60 and 100 psi are often used to inflate catheter balloons and, in the case of a rupture, the escaping inflation fluid as well as any dislodged pieces of balloon membrane can impact sensitive body tissue it comes in contact with. Any residual pieces of membrane that are not recovered can also pose a further danger to the patient.

In the event of a balloon failure taking place during a medical procedure, the catheter generally has to be removed from the patient and a new catheter percutaneously passed into the procedure site. Removal of a catheter with a ruptured balloon attached can sometime prove difficult and potentially dangerous because fragments of the ruptured balloon can be stripped away from the catheter as it is being withdrawn.

### SUMMARY OF THE INVENTION

It is a primary object of the present invention to improve balloon catheters.

It is a further object of the present invention to protect a patient from harm in the event a balloon that is attached to the distal end of a catheter ruptures during a medical procedure.

A still further object of the present invention is to safely contain the inflation fluid as well as balloon fragments of a balloon catheter in the event the balloon fails during a medical procedure.

Yet another object of the present invention is to prevent the interruption of an in-process medical procedure in the event an inflated balloon of a balloon catheter ruptures during a medical procedure.

These and other objects of the present invention are attained by means of a balloon catheter that has an outer balloon affixed to the distal end of the catheter insertion tube. A second inner balloon is similarly affixed to the insertion tube and is entirely enclosed within the outer balloon. A vacuum pump is connected to the outer balloon through the insertion tube and is arranged to evacuate the atmosphere of the outer balloon. A pump is connected to the inner balloon which is arranged to inflate the inner balloon to a desired pressure. In the event, the inner balloon is ruptured during inflation, the high pressure inflation fluid is released into the outer balloon and is thus contained therein along with any of the inner balloon fragments that might be dislodged because of the rupture. Finally, in certain cases, the medical procedure can proceed without interruption after a rupture by simply inflating the outer balloon to a desired operational level.

### BRIEF DESCRIPTION OF THE DRAWINGS

For a better understanding of these and other objects of the present invention, reference will be made to the following detailed description of the invention that should be read in association with the accompanying drawings, wherein:

FIG. 1 is a side elevation illustrating a balloon catheter embodying the teachings of the present invention;

FIG. 2 is an enlarged sectional view taken along lines 2-2 in FIG. 1 showing the inner and outer balloons in a semi-inflated state; and

FIG. 3 is an enlarged sectional view taken along lines 3-3 in FIG. 1 showing the inner balloon fully inflated within the outer balloon.

### DETAILED DESCRIPTION

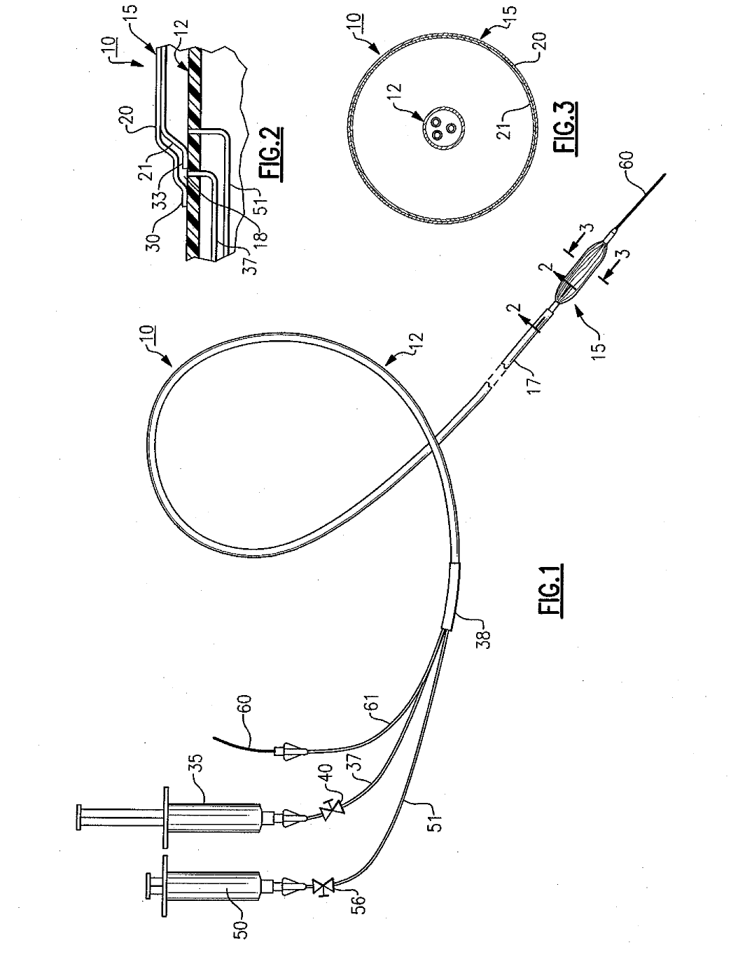

A balloon catheter, generally referenced 10, that embodies the present invention, is illustrated in FIG. 1, The catheter includes a hollow insertion tube 12 that is fabricated of a plastic material having sufficient flexibility such that it can be percutaneously inserted into a desired body cavity via a blood vessel. A balloon assembly, generally referenced 15 is affixed to the distal end 17 of the insertion tube. The assembly consists of a first cylindrical outer balloon 20 that surrounds the insertion tube and a second cylindrical inner balloon 21 that also surrounds the insertion tube and is completely contained within the outer balloon. Each of the balloons is made of a thin wall membrane that is capable of expanding when inflated with a suitable fluid which can be a gas, vapor or fluid. In most cases, air is used to inflate most balloons used in the art. The axial length of the outer balloon is slightly longer than that of the inner balloon and the inner balloon is centered within the outer balloon to provide a space 18 at either end of the assembly between the balloons. The balloons can be fabricated of any one of many well known copolymers that are used in the art. Preferably, both balloons are made of the same material.

The thin walls of the two superimposed balloons, when deflated, provides an extremely compact package upon the distal insertion tube which can pass with relative ease through a blood vessel into a desired treatment zone. The size of the dual balloon package is about the same size as that of a single balloon package and thus does not present any additional problem during insertion and removal of the catheter. The opposite ends of the outer balloons each contain a cuff 30 (FIG. 2) that surrounds the insertion tube and are affixed thereto to establish an air tight joint between the two cuffs and the tube. Similarly, the two ends of the inner balloon each contain a cuff 33 that is affixed to the insertion tube to provide an air tight joint therebetween.

With further reference to FIG. 2, the outer balloon 20 is connected to a syringe 35 capable of evacuating the balloon via a lumen 37. One end of the lumen is coupled to the syringe and passes through the insertion tube to a point beneath the balloon package. The terminal end 38 of the lumen is passed through the insertion tube into the space 18 between the two balloons and the connection rendered air tight by any suitable means. A shut off valve 40 is mounted in the proximal end of the lumen which when placed in an open position permits the syringe to evacuate the outer balloon. Placing the valve in a closed position, in turn, holds the vacuum within the balloon and thus maintains the balloon in an evacuated condition.

The inner balloon is similarly connected to a syringe 50 by means of a second lumen 51. Again, the syringe is coupled to the proximal end of the lumen and the lumen is passed through the insertion tube to a point below the inner balloon. The distal end of the lumen is passed through the insertion tube into the inner balloon and the connection between the lumen and the insertion tube is closed to form an air tight joint therebetween. A second stop valve 56 is mounted in the proximal end of lumen 51. When the valve is placed in an open position, the syringe is able to deliver sufficient air into the inner balloon to completely inflate the balloon. Closing the valve will prevent air from escaping from the balloon and thus maintain the balloon in an inflated condition.

In operation, before passing the catheter into a blood vessel, the stop valve 56 that controls inflation of the inner balloon is opened as well as the stop valve 40 that controls the evacuation of the outer balloon thus allowing the balloons to be wrapped about the insertion tube into a tight package and the outer balloon is evacuated and valve 40 is closed. Once the balloon package is properly positioned in the treatment site, the inner balloon is inflated. The outer balloon at this time provides little resistance to the expanding inner balloon and simply rides upwardly on the outer balloon until it reaches full inflation whereupon stop valve 56 is closed. If for any reason the inner balloon should fail while fully or partially inflated, the air contained within the inner balloon with be discharged into the slightly larger outer balloon and be fully contained therein. Any fragments of the inner balloon that might breakaway will also be captured within the outer balloon and eventually be safely removed from the patient when the catheter is removed.

The addition of the second balloon to the catheter consumes little additional space and sufficient room is made available within the insertion tube for a third lumen 61 through which a guide wire can pass.

In many cases, failure of a balloon while a medical procedure is in process results in the procedure being terminated and the catheter being removed from the patient and a new procedure having to be initiated. This is not only time consuming but also potentially dangerous and uncomfortable for the patient. The problems associated with a balloon failure during a medical procedure can be avoided by use of this dual balloon arrangement. If such a failure does occur during a procedure, the air escaping from the ruptured inner balloon will partially fill or almost completely fill the outer balloon. If necessary, the syringe servicing the outer balloon can be used to fully inflate the outer balloon through proper operation of the associated stop valve and the procedure carried on to completion.

While the invention has been described with reference to preferred embodiments, it will be understood by those skilled in the art that various changes may be made and equivalents may be substituted for elements thereof to adapt to particular situations without departing from the scope of the invention. Therefore, it is intended that the invention not be limited to the particular embodiments disclosed as the best mode contemplated for carrying out this invention, but that the invention will include all embodiments falling within the scope and spirit of the appended claims.

1. A balloon catheter for protecting a patient from harm that includes:
an insertion tube having a distal end and a proximal end;
an outer balloon that surrounds the distal end of said insertion tube;
an inner balloon that surrounds the distal end of said insertion tube said inner balloon being fully contained inside said outer balloon;
evacuating means for drawing fluid from said outer balloon to evacuate said outer balloon; and
inflation means for independently inflating said inner balloon inside said evacuated outer balloon.

2. The catheter of claim 1, that further includes a first lumen contained within said insertion tube for placing said evacuating means in fluid flow communication with said outer balloon and a second lumen also contained within said insertion tube for placing the inflation means in fluid flow communication with said inner balloon.

3. The catheter of claim 2, wherein said outer balloon and said inner balloon are cylindrical shaped and the axial length of the outer balloon is greater than that of the inner balloon to provide a space between the two balloons.

4. The catheter of claim 3, wherein said first lumen enters the outer balloon through said insertion tube within said space between said balloons.

5. The catheter of claim 4, wherein said first and second lumens each contain a valve means for opening and closing said lumens so that the flow of air into and out of said balloons can be manually controlled.

6. The catheter of claim 4, wherein said evacuating means is a first syringe that is connected to said first lumen.

7. The catheter of claim 7, wherein said inflation means is a second syringe that is connected to said second lumen.

8. A method of protecting a patient during a medical procedure involving a balloon catheter that includes the steps of:
attaching an outer balloon to the distal end of an insertion tube of a catheter to establish an air tight joint between said outer balloon and said insertion tube;
attaching an inner balloon to the distal end of said insertion tube to establish an air tight joint between the said inner balloon and said insertion tube, said inner balloon being fully contained within said outer balloon;
inserting the distal end of the insertion tube percutaneously into a treatment site;
evacuating the outer balloon; and
inflating said inner balloon.
